(19) United States
(12) Patent Application Publication
(10) Pub. No.: US 2025/0006410 A1
(43) Pub. Date: Jan. 2, 2025

(54) Magnetic Nanoparticles

(71) Applicant: The University of Sydney, Sydney (AU)

(72) Inventors: Gurvinder Singh; Hala Zreiqat, Sydney, NSW (AU)

(73) Assignee: The University of Sydney, Sydney (AU)

(21) Appl. No.: 18/688,676

(22) PCT Filed: Sep. 1, 2022
(86) PCT No.: PCT/AU2022/051069

§ 371 Date: Mar. 1, 2024

(30) Foreign Application Priority Data
Sep. 1, 2021 (AU) 2021902833

(51) Int. Cl. H01F 1/00; A61K 41/00; B82Y 5/00; B82Y 25/00; B82Y 40/00; C01G 49/00; C01G 51/00; H01F 1/34

(52) U.S. Cl. CPC H01F 1/0045; A61K 41/0052; C01G 49/0072; C01G 51/40; H01F 1/344; B82Y 5/00; B82Y 25/00; B82Y 40/00; C01P 2002/72; C01P 2002/85; C01P 2004/04; C01P 2004/64; C01P 2004/88; C01P 2006/42

(57) ABSTRACT

The present disclosure relates to magnetic nanoparticles having a core-multishell structure comprising at least two shells, and methods for their preparation.

### FIELD

The present disclosure relates to magnetic nanoparticles having a core-multishell structure comprising at least two shells. The present disclosure also relates to methods of preparing magnetic nanoparticles having a core-multishell structure comprising at least two shells. The present disclosure further relates to methods of using the magnetic nanoparticles.

### BACKGROUND

Magnetic nanoparticles are small particles typically around 1 to 100 nm in size and have unique magnetic properties. Due to their small size and attractive properties, there is interest in developing magnetic nanoparticles for a wide range of potential applications, including biomedical, energy storage and information storage applications.

Many of the magnetic nanoparticles developed to date are single phase magnetic nanoparticles composed of a single magnetic material, and bimagnetic “core-shell” nanoparticles having a core composed of one magnetic material surrounded by a single shell composed of another magnetic material. Although some trimagnetic nanoparticles have been prepared, there are challenges in their production. Some of these challenges include controlling the size and shape of each magnetic phase, particularly shell thickness, as well as avoiding inter phase contamination, which may influence the crystallinity and phase purity of both individual shell structures and interphase structures of the nanoparticles. These issues can make it difficult to reliably prepare such magnetic nanoparticles and to determine and tune their magnetic properties for a desired application.

Another challenge in the development of magnetic nanoparticles is that reducing magnetic materials to nanoscale size can affect their fundamental magnetic properties, which may limit the use of magnetic nanoparticles in certain applications. For example, typically when the size of magnetic nanoparticles decrease, they start to lose their ability to behave like a ferromagnetic material at room temperature in the absence of an applied magnetic field and instead behave like superparamagnetic materials. This may prevent the use of such magnetic nanoparticles for applications that require them to have ferromagnetic properties, such as magnetic hyperthermia, energy storage and conversion, separation, and data storage applications.

Accordingly, there is a need for alternative magnetic nanoparticles and methods for their preparation, which may advantageously address one or more of the above identified problems.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY

In one aspect, the present disclosure provides a magnetic nanoparticle having a core-multishell structure, said core-multishell structure comprising:
a core, said core comprising a first magnetic material;
a first shell, said first shell surrounding the core, said first shell comprising a second magnetic material;
a second shell, said second shell surrounding the first shell, said second shell comprising a third magnetic material; and
optionally one or more further shells, said one or more further shells surrounding an immediately preceding shell, said one or more further shells each independently comprising a magnetic material;
   wherein the second magnetic material has a different coercivity (H_{C}) to the first magnetic material, the third magnetic material has a different H_{C} to the second magnetic material, and the optional one or more further shells each comprise magnetic material having a different H_{C} to magnetic material in an immediately preceding shell.

In some embodiments, the first shell, the second shell and each optional further shell alternate between comprising magnetic material having a lower or higher H_{C} than an immediately preceding shell (or, in the case of the first shell, the core).

In some embodiments, the first magnetic material and the third magnetic material are the same.

In some embodiments, the magnetic nanoparticle further comprises a third shell comprising a fourth magnetic material, wherein the fourth magnetic material has a different H_{C} to the third magnetic material.

In some embodiments, the first magnetic material and the second magnetic material independently comprise one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide.

In some embodiments, the first magnetic material is a hard phase magnetic material, preferably one or more of cobalt ferrite, barium ferrite and strontium ferrite.

In some embodiments, the magnetic nanoparticle has a core-multishell structure selected from:
CoFe₂O₄@MnFe₂O₄@CoFe₂O₄ (core@shell@shell)
CoFe₂O₄@BaFe₁₂O₁₉@CoFe₂O₄ (core@shell@shell)
BaFe₁₂O₁₉@CoFe₂O₄@BaFe₁₂O₁₉ (core@shell@shell)
CoFe₂O₄@SrFe₁₂O₁₉@CoFe₂O₄ (core@shell@shell)
SrFe₁₂O₁₉@CoFe₂O₄@SrFe₁₂O₁₉ (core@shell@shell)
CoFe₂O₄@MnFe₂O₄@CoFe₂O₄@MnFe₂O₄ (core@shell@shell@shell)
CoFe₂O₄@BaFe₁₂O₁₉@CoFe₂O₄@BaFe₁₂O₁₉ (core@shell@shell@shell)

In some embodiments of the magnetic nanoparticle, one or more of the following apply:
the core has an average diameter of from about 5 nm to about 15 nm, preferably about 6 to about 10 nm;
each shell independently has an average thickness of from about 1 nm to about 5 nm, preferably about 2 nm to about 4 nm;
the magnetic nanoparticle has an average diameter of less than about 40 nm, preferably less than about 30 nm, more preferably less than about 25 nm, even more preferably less than about 20 nm.

In some embodiments, the magnetic nanoparticle is ferromagnetic.

In some embodiments, the magnetic nanoparticle exhibits ferromagnetism at room temperature without application of an external magnetic field.

In some embodiments, the magnetic nanoparticle further comprises one or more functional moieties attached to the outermost shell of the core-multishell structure. Each of the one or more functional moieties may be independently attached by a covalent bond or a non-covalent bond (eg by electrostatic interaction).

In some embodiments, the magnetic nanoparticle has a specific absorption rate (SAR) of at least about 20 W/g, measured at a field strength of 15 V at a field frequency of 176 KHz.

In another aspect, the present disclosure provides a method of preparing a magnetic nanoparticle having a core-multishell structure, the method comprising:
providing a nanoparticle core comprising a first magnetic material;
heating a first mixture comprising the nanoparticle core, one or more second magnetic material precursors and one or more surfactants in a dispersing medium to a temperature not exceeding about 290° C. such that a first shell comprising a second magnetic material is deposited on the nanoparticle core to provide a core-shell nanoparticle comprising a single shell; and
heating a second mixture comprising the core-shell nanoparticle, one or more third magnetic material precursors and one or more surfactants in a dispersing medium to a temperature not exceeding about 290° C. such that a second shell comprising a third magnetic material is deposited on the first shell to provide a core-multishell nanoparticle comprising two shells;
wherein the second magnetic material has a different coercivity (H_{C}) to the first magnetic material, and the third magnetic material has a different H_{C} to the second magnetic material.

In some embodiments, the method further comprises:
heating a third mixture the core-multishell nanoparticle comprising two shells, one or more fourth magnetic material precursors and one or more surfactants in a dispersing medium to a temperature not exceeding about 290° C. such that a third shell comprising a fourth magnetic material is deposited on the second shell to provide a core-multi-shell nanoparticle comprising three shells;
   wherein the third magnetic material has a different H_{C} to the second magnetic material.

In some embodiments, the method further comprises:
(i) heating a further mixture comprising the core-multishell nanoparticle, one or more further magnetic material precursors and one or more surfactants in a dispersing medium to a temperature not exceeding about 290° C. such that a further shell comprising a further magnetic material is deposited on the outermost shell of the core-multishell nanoparticle; and
(ii) optionally repeating step (i) one or more times;
   to provide one or optionally more further shells each independently comprising a further magnetic material on the core-multishell nanoparticle;
   wherein the one or optionally more additional shells each have a different H_{C} to the preceding shell.

In some embodiments, each heating step independently comprises heating to a temperature not exceeding the boiling point of the respective dispersing medium.

In some embodiments, each heating step independently comprises heating at a rate of from about 3° C./min to about 10° C./min, preferably at a rate of about 5° C./min.

In some embodiments, each heating step independently comprises heating at the temperature for a duration of from about 20 minutes to about 45 minutes, preferably for a duration of about 30 minutes.

In some embodiments, the first magnetic material comprises one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide.

In some embodiments, the first magnetic material is a hard phase magnetic material, preferably one or more of cobalt ferrite, barium ferrite and strontium ferrite.

In some embodiments, each of the one or more respective magnetic material precursors independently comprise an iron precursor and one or more of a cobalt precursor, a barium precursor, a strontium precursor, a manganese precursor and a nickel precursor. In these embodiments, the molar ratio of the iron precursor to the one or more of a cobalt precursor, a barium precursor, a strontium precursor, a manganese precursor and a nickel precursor may be about 1:1 to about 3:1, preferably about 2:1.

In some embodiments of the method, one or more of the following apply:
the molar ratio of nanoparticle core to the one or more second magnetic material precursors is about 1:3 to about 1:1, preferably about 1:2;
the molar ratio of the core-shell nanoparticle comprising a single shell to the one or more third magnetic material precursors is about 1:3 to about 1:1, preferably about 1:2;
the molar ratio of the core-multishell nanoparticle comprising two shells to the one or more third magnetic material precursors is about 1:3 to about 1:1, preferably about 1:2;
the molar ratio of the core-multishell nanoparticle to the one or more further magnetic material precursors is about 1:3 to about 1:1, preferably about 1:2.

In some embodiments, each of the dispersing mediums independently comprise one or more non-polar organic liquids.

In some embodiments, each of the dispersing mediums independently have a minimum boiling point of greater than about 180° C.

In some embodiments, each of the dispersing mediums independently comprise one or more of benzyl ether, phenyl ether, hexadecane and octadecene, preferably benzyl ether.

In some embodiments, each of the dispersing mediums independently consist of a single organic liquid, preferably benzyl ether.

In some embodiments, each of the one or more surfactants independently comprise one or more cationic surfactants, anionic surfactants and non-ionic surfactants comprising at least one saturated or unsaturated hydrocarbon chain greater than 8 carbon atoms.

In some embodiments, each of the one or more surfactants independently consist of a single surfactant, preferably oleic acid.

In some embodiments of the method, one or more of the following apply:
the nanoparticle core has an average diameter of from about 5 nm to about 15 nm, preferably about 6 to about 10 nm;
each shell independently has an average thickness of from about 1 nm to about 5 nm, preferably about 2 nm to about 4 nm;
the magnetic nanoparticle has an average diameter of less than about 40 nm, preferably less than about 30 nm, more preferably less than about 25 nm, even more preferably less than about 20 nm.

In some embodiments, the method further comprises functionalising the outermost shell of the outermost shell of the core-multishell nanoparticle with one or more functional moieties.

The magnetic nanoparticle prepared by the method may be the magnetic nanoparticle as described herein.

In another aspect, the present disclosure provides the magnetic nanoparticle prepared by the method described herein.

In another aspect, the present disclosure provides a method of preparing a functionalised nanoparticle, the method comprising:
providing the magnetic nanoparticle having a core-multishell structure described herein or prepared by the method described herein, and
functionalising the outermost shell of the core-multishell structure with one or more functional moieties.

In another aspect, the present disclosure provides a method of magnetic hyperthermia comprising:
administering the magnetic nanoparticle described herein or prepared by the method described herein to a patient, and
exposing the magnetic nanoparticle to an alternating magnetic field.

The present disclosure may provide one or more of the following advantages:
magnetic nanoparticles having improved magnetic properties, such as high thermal stability at room temperature;
magnetic nanoparticles which can exhibit ferromagnetism at room temperature in the absence of an applied magnetic field;
simple and reproducible methods for preparing core-multishell magnetic nanoparticles that comprise two or more shells;
ability to control of the size and shape of the core and shell structures, particularly shell thickness, as well as the overall structure of the magnetic nanoparticles;
ability to tailor properties of the magnetic nanoparticles for various applications.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Schematic representation illustrating the formation of a magnetic nanoparticle having a core-multishell structure described herein.

FIG. 2. Transmission electron microscopy (TEM) images of CoFe₂O₄ nanoparticle seeds (core) (FIG. 2A) and CoFe₂O₄@MnFe₂O₄ (core@shell) (FIG. 2B), CoFe₂O₄@MnFe₂O₄@CoFe₂O₄ (core@shell@shell) (FIG. 2C) and CoFe₂O₄@MnFe₂O₄@CoFe₂O₄@MnFe₂O₄ (core@shell@shell@shell) (FIG. 2D) nanoparticles. The inset of FIG. 2A is a high resolution TEM image of the CoFe₂O₄ seed sample.

FIG. 3. Energy dispersive spectrometer (EDS) elemental mapping of CoFe₂O₄@MnFe₂O₄@CoFe₂O₄@MnFe₂O₄ magnetic nanoparticles. Iron, cobalt and manganese appear white in their respective panel.

FIG. 4. Comparative X-ray diffraction (XRD) patterns of CoFe₂O₄ nanoparticle seeds and CoFe₂O₄@MnFe₂O₄, CoFe₂O₄@MnFe₂O₄@CoFe₂O₄ and CoFe₂O₄@MnFe₂O₄@CoFe₂O₄@MnFe₂O₄ nanoparticles. The traces are superimposable.

FIG. 5. Coercivity (H_{C}) and saturation magnetisation (M₅) results for CoFe₂O₄ nanoparticle seeds and CoFe₂O₄@MnFe₂O₄, CoFe₂O₄@MnFe₂O₄@CoFe₂O₄ and CoFe₂O₄@MnFe₂O₄@CoFe₂O₄@MnFe₂O₄ nanoparticles at 300K. Each line is labelled from the key by an arrow.

FIG. 6. Blocking temperature (T_{B}) of CoFe₂O₄ nanoparticle seeds and CoFe₂O₄@MnFe₂O₄, CoFe₂O₄@MnFe₂O₄@CoFe₂O₄ and CoFe₂O₄@MnFe₂O₄@CoFe₂O₄@MnFe₂O₄ nanoparticles determined by zero field cooling and field cooling (ZFC/FC) curves.

FIG. 7. Specific absorption rate (SAR) results for functionalised CoFe₂O₄@MnFe₂O₄ and functionalised CoFe₂O₄@MnFe₂O₄@CoFe₂O₄@MnFe₂O₄ magnetic nanoparticles at different voltages at a fixed field frequency of 176 Hz, compared with literature Fe(Co)—Au nanoparticles.

### DETAILED DESCRIPTION OF THE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure.

Although any methods and materials similar or equivalent to those described herein can also be used in the practice or testing of the present disclosure, the preferred methods and materials are now described.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

As used in the specification and the appended claims, the singular forms ‘a’, ‘an’ and ‘the’ include plural referents unless otherwise specified. Thus, for example, reference to ‘a magnetic material’ may include one or more magnetic material(s) and reference to ‘a magnetic material precursor’ may include at least one magnetic material precursor, and the like.

As used herein, except where the context requires otherwise, the term ‘comprise’ and variations of the term, such as ‘comprising’, ‘comprises’ and ‘comprised’, are not intended to exclude further additives, components, integers or steps.

Unless specifically stated or obvious from context, as used herein, the term ‘about’ is understood as within a range of normal tolerance in the art, for example within two standard deviations of the mean. ‘About’ can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term ‘about’.

Ranges provided herein are understood to be shorthand for all of the values, including non-integer values, within the range. For example, a range of 1 to 50 is understood to include any number, combination of numbers, or sub-range from the group consisting 1, 1.01, 2, 2.2, 3, 3.45, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

It will be understood that the disclosure described and defined in this specification extends to all alternative combinations of two or more of the individual features mentioned or evident from the text or drawings. All of these different combinations constitute various alternative aspects of the disclosure.

Context allowing, it is intended that any embodiment described herein may be combined with any other embodiment.

Any methods provided herein can be combined with one or more of any of the other methods provided herein.

### Magnetic Nanoparticles

The present disclosure provides a magnetic nanoparticle having a core-multishell structure, also described as an onion structure, where the term “multishell” will be understood to refer to at least two shells. The core-multishell structure comprises:
a core, said core comprising a first magnetic material;
a first shell, said first shell surrounding the core, said first shell comprising a second magnetic material;
a second shell, said second shell surrounding the first shell, said second shell comprising a third magnetic material; and
optionally one or more further shells, said one or more further shells surrounding an immediately preceding shell, said one or more further shells each independently comprising a magnetic material.

Each shell (or layer) comprises magnetic material that is different to the immediately preceding shell (or in the case of the first shell, the core). Therefore, the second magnetic material has a different coercivity (H_{C}) to the first magnetic material, the third magnetic material has a different H_{C} to the second magnetic material, and the optional one or more further shells each comprise magnetic material having a different H_{C} to magnetic material in an immediately preceding shell. The term “coercivity” (denoted H_{C}) will be understood to refer to the measure of the ability of a ferromagnetic material to withstand an external magnetic field without becoming demagnetised. The term “different coercivity” in the context of the present disclosure will be understood to mean that the magnetic material has a coercivity that is higher or lower than the coercivity of the magnetic material immediately preceding shell (or in the case of the first shell, the core).

The first shell surrounds the core, that is, the first shell is a layer on, including in direct contact with, the core. Similarly, the second shell surrounds the first shell, that is, the second shell in a layer on, including in direct contact with, the first shell, and so on for the third shell (if present) and optional further one or more shells. In the context of the present disclosure, the term “surrounds” will be understood to mean that the shell completely covers the immediately preceding shell (or in the case of the first shell, the core). The existence of the core-multishell structure of the magnetic nanoparticle may be determined by methods known in the art, for example by X-ray photoelectron microscopy (XPS).

Without wishing to be bound by theory, it is hypothesised that the exchange coupling between two magnetic materials having different coercivity may affect the thermal stability of the magnetic moment of the magnetic nanoparticle, and therefore the simultaneous couplings between the magnetic materials contacting each other in the core-multishell structure of the magnetic nanoparticle disclosed herein may influence the overall magnetic properties of the magnetic nanoparticle. The present inventors have found that providing a core-multishell structure in which each shell comprises magnetic material which is in contact with one or two shells (or, in the case of the first shell, in contact with the core and the second shell) each comprising a magnetic material having a different coercivity may allow for tuning of the magnetic properties of the magnetic nanoparticle. Advantageously, the multiple exchange couplings at the interface between the different magnetic materials may provide the core-multishell magnetic nanoparticles disclosed herein with improved magnetic properties compared to their individual magnetic material counterparts and core-shell structures having only a single shell.

The magnetic nanoparticle may comprise a third shell comprising a fourth magnetic material, wherein the fourth magnetic material has a different H_{C} to the third magnetic material. The magnetic nanoparticle may optionally comprise one or more further shells in addition to the third shell.

Accordingly, in some embodiments, magnetic nanoparticle comprises a core-multishell structure comprising:
a core, said core comprising a first magnetic material;
a first shell, said first shell surrounding the core, said first shell comprising a second magnetic material;
a second shell, said second shell surrounding the first shell, said second shell comprising a third magnetic material;
a third shell, said third shell surrounding the second shell, said third shell comprising a fourth magnetic material; and
optionally one or more further shells, said one or more further shells surrounding an immediately preceding shell, said one or more further shells each independently comprising a magnetic material;
   wherein the second magnetic material has a different coercivity (H_{C}) to the first magnetic material, the third magnetic material has a different H_{C} to the second magnetic material, the fourth magnetic material has a different H_{C} to the second magnetic material, and the optional one or more further shells each comprise magnetic material having a different H_{C} to magnetic material in an immediately preceding shell.

In some embodiments, the core-multishell structure of the magnetic nanoparticle comprises two or more shells. In some embodiments, the core-multishell structure of comprises three or more shells.

The first shell, the second shell, the third shell (if present) and each optional further shell alternate between comprising magnetic material having a lower or higher H_{C} than an immediately preceding shell (or, in the case of the first shell, the core). By way of example, the second magnetic material may have a lower H_{C} than the first magnetic material, the third magnetic material may have a higher H_{C} than the second magnetic material, the fourth magnetic material (if present) may have a higher H_{C} than the third magnetic material, and so on. Alternatively, the second magnetic material may have a higher H_{C} than the first magnetic material, the third magnetic material may have a lower H_{C} than the second magnetic material, the fourth magnetic material (if present) may have a higher H_{C} than the third magnetic material, and so on.

The core and the second shell may both comprise the same magnetic material. Accordingly, in some embodiments, the first magnetic material and the third magnetic material are the same.

In embodiments where the magnetic nanoparticle comprises a third shell, the first shell and the third shell may both comprise the same magnetic material. Accordingly, in some embodiments, the second magnetic material and the fourth magnetic material are the same.

The magnetic material of the core and each shell may be any suitable magnetic material. Examples of suitable magnetic materials include cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide. Each magnetic material may independently be a ferromagnetic material. In some embodiments, the first magnetic material comprises one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide. In some embodiments, the second material comprises one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide. In some embodiments, the third material comprises one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide. In embodiments where the magnetic nanoparticle comprises a third shell, the fourth magnetic material may comprise one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide. The magnetic material of each optional further shell may independently comprise one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide.

It will be appreciated that magnetic materials may be classified as “soft”, “semi-hard” or “hard” in terms of their magnetic properties based on their magnetic coercivity (H_{C}). Generally, a soft magnetic material will have a low coercivity and a hard magnetic material will have a high coercivity. In the context of the present disclosure, “hard phase” magnetic materials will be understood to encompass magnetic materials classified as “hard” as well as “semi-hard” magnetic materials. Examples of soft phase magnetic materials include nickel ferrite, manganese ferrite, manganese-zinc ferrite, zinc ferrite, zinc-iron ferrite, zinc-nickel-ferrite and iron oxide. Examples of hard phase magnetic materials include cobalt ferrite, barium ferrite and strontium ferrite.

The first magnetic material (ie the magnetic material of the core) may comprise one or more soft phase magnetic materials or one or more hard phase magnetic materials. The first magnetic material may be suitably selected based on the intended application of the magnetic nanoparticle and the properties of the magnetic nanoparticle required for that application. It will be appreciated that a magnetic nanoparticle comprising a soft phase core magnetic material will have different magnetic properties to a magnetic nanoparticle comprising a hard phase core magnetic material, which may be due to the overall amounts (wt %) or ratios of the hard to soft phases present in the respective magnetic nanoparticles. Typically, the higher the amount of hard phase magnetic material, the higher the coercivity of the magnetic nanoparticle at room temperature.

In preferred embodiments, the first magnetic material comprises one or more hard phase magnetic materials. The one or more hard phase magnetic materials may be selected from one or more of cobalt ferrite, barium ferrite and strontium ferrite.

Accordingly, in some embodiments, the magnetic nanoparticle comprises a core-multishell structure comprising:
a core, said core comprising a first magnetic material;
a first shell, said first shell surrounding the core, said first shell comprising a second magnetic material;
a second shell, said second shell surrounding the first shell, said second shell comprising a third magnetic material; and
optionally one or more further shells, said one or more further shells surrounding an immediately preceding shell, said one or more further shells each independently comprising a magnetic material;
   wherein the second magnetic material has a different coercivity (H_{C}) to the first magnetic material, the third magnetic material has a different H_{C} to the second magnetic material, and the optional one or more further shells each comprise magnetic material having a different H_{C} to magnetic material in an immediately preceding shell; and wherein the first magnetic material comprises one or more hard phase magnetic materials.

In embodiments where the first magnetic material comprises one or more hard phase magnetic materials, the second magnetic material may have a lower coercivity to the first magnetic material. By way of example, in embodiments where the first magnetic material comprises cobalt ferrite, the second magnetic material may comprise one or more of nickel ferrite, manganese ferrite, manganese-zinc ferrite, zinc ferrite, zinc-iron ferrite, zinc-nickel-ferrite and iron oxide. In another example, in embodiments where the first magnetic material comprises barium ferrite, the second magnetic material may comprise one or more of cobalt ferrite, nickel ferrite, manganese ferrite, manganese-zinc ferrite, zinc ferrite, zinc-iron ferrite, zinc-nickel-ferrite and iron oxide. In another example, in embodiments where the first magnetic material comprises strontium ferrite, the second magnetic material may comprise one or more of cobalt ferrite, nickel ferrite, manganese ferrite, manganese-zinc ferrite, zinc ferrite, zinc-iron ferrite, zinc-nickel-ferrite and iron oxide.

In embodiments where the first magnetic material comprises one or more hard phase magnetic materials, the second magnetic material have a higher coercivity to the first magnetic material. By way of example, in embodiments where the first magnetic material comprises cobalt ferrite, the second magnetic material may comprise one or more of barium ferrite and strontium ferrite.

In some embodiments, the magnetic nanoparticle comprises a core-multishell structure selected from:
CoFe₂O₄@MnFe₂O₄@CoFe₂O₄ (core@shell@shell)
CoFe₂O₄@BaFe₁₂O₁₉@CoFe₂O₄ (core@shell@shell)
BaFe₁₂O₁₉@CoFe₂O₄@BaFe₁₂O₁₉ (core@shell@shell)
CoFe₂O₄@SrFe₁₂O₁₉@CoFe₂O₄ (core@shell@shell)
SrFe₁₂O₁₉@CoFe₂O₄@SrFe₁₂O₁₉ (core@shell@shell)
CoFe₂O₄@MnFe₂O₄@CoFe₂O₄@MnFe₂O₄ (core@shell@shell@shell)
CoFe₂O₄@BaFe₁₂O₁₉@CoFe₂O₄@BaFe₁₂O₁₉ (core@shell@shell@shell)

The magnetic nanoparticle may comprise one or more functional moieties attached to the outermost shell (ie the surface) of the magnetic nanoparticle, including functional moieties known in the art. Accordingly, the magnetic nanoparticle may be a functionalised magnetic nanoparticle. The functional moieties can be used to modify one or more properties of the magnetic nanoparticle and/or impart the magnetic nanoparticle with additional functionality. The use of multiple functional moieties provides a multifunctional nanoparticle. Examples of suitable functional moieties include polymers, such as polyethylene glycol (PEG), polyvinyl alcohol (PVA), polyvinylpyrrolidone (PVP) and dextran; targeting moieties that can be used to target certain biological sites or tissues; and therapeutic cargo, including small molecule drugs and macromolecular drugs. Small molecule drugs may include any suitable organic compound having a low molecular weight (less than 900 Daltons) that can regulate a biological process to treat a particular disease, for example doxorubicin. Macromolecular drugs may include large molecules (molecular weight more than about 900 Daltons) such as proteins, polysaccharides and nucleic acids and that can regulate a biological process to treat a particular disease, for example mRNA. The therapeutic cargo may be delivered and/or released at the target site of the disease.

The functional moieties may be suitably selected based on an intended use of the magnetic nanoparticle. By way of example, coating a magnetic nanoparticle with a polymer such as PEG may be useful for one or more of the following: increasing hydrophilicity, improving biocompatibility, improving stability, and improving dispersion (or preventing or reducing aggregation) of the nanoparticles in a polar solvent. Functionalised nanoparticles may be useful in certain applications including biomedical applications such as magnetic hyperthermia and bioimaging, and targeted drug delivery. For example, coating a magnetic nanoparticle with PEG may prevent or reduce unspecific adsorption of protein molecules present in the blood. In another example, functionalising a magnetic particle with a targeting moiety may allow the magnetic nanoparticle to specifically target a biological site or tissue as required for the treatment of a certain disease. It will be appreciated that the choice of targeting moiety will depend on the disease to be treated, for example suitable targeting moieties for targeting breast cancer may include folic acid and transferrin molecules. In a further example, functionalising a magnetic particle with a therapeutic cargo may allow the magnetic nanoparticle to deliver and/or release the therapeutic cargo at the target site of a certain disease.

The one or more functional moieties may each be independently attached to the magnetic nanoparticle by a covalent bond or a non-covalent bond (eg by electrostatic interaction). Accordingly, in some embodiments, the magnetic nanoparticle described herein comprises one or more functional moieties attached by a covalent bond. Examples of covalent bonds include an amide or carbonyl bond or any other suitable covalent bond for attaching the functional moiety to the surface of the core-multishell nanoparticle. Additionally, or alternatively, in some embodiments, the magnetic nanoparticle comprises one or more functional moieties attached by a non-covalent bond, preferably by electrostatic interaction. It will be appreciated that the type of attachment may depend on the functional moiety to be attached.

The magnetic nanoparticle may be any suitable shape. Generally, the nanoparticles may be spherical, although this term encompasses irregularly shaped particles which are still reasonably defined by a sphere, and also encompasses particles which may have one or more flat facets (eg nanocubes).

The magnetic nanoparticle may have an average diameter of less than about 50 nm, less than about 40 nm, preferably less than about 30 nm, more preferably less than about 25 nm, even more preferably less than about 20 nm. In some embodiments, the magnetic nanoparticle has an average diameter of from about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 32 nm, about 34 nm, about 36 nm, about 38 nm, about 40 nm, about 45 nm, or about 50 nm. Any minimum and maximum value may be combined to form a range provided the average diameter is within 7 nm to 50 nm, for example about 7 nm to about 30 nm or from about 10 nm to about 25 nm. The average diameter may be determined by methods known in the art, for example measured from transmission electron microscopy (TEM) images. The term “average” will be understood to mean an average value as determined from a representative number of individual nanoparticles in one or more samples.

The core of the magnetic nanoparticle may an average diameter of from about 5 nm to about 15 nm, for example an average diameter of about 5 nm, about 6 nm, about 7 nm, about 8 nm, about 9 nm, about 10 nm, about 11 nm, about 12 nm, about 13 nm, about 14 nm, or about 15 nm. Any minimum and maximum value may be combined to form a range provided the average diameter is within 5 nm to 15 nm, for example from about 6 nm to about 10 nm. The average diameter may be determined by methods known in the art, for example measured from transmission electron microscopy (TEM) images. The term “average” will be understood to mean an average value as determined from a representative number of individual nanoparticles in one or more samples.

Each shell of the magnetic nanoparticle may independently have a thickness of from about 1 nm to about 5 nm, for example a thickness of about 1 nm, about 2 nm, about 3 nm, about 4 nm or about 5 nm. Any minimum and maximum value may be combined to form a range provided the thickness is within 1 nm to 5 nm, for example 2 nm to 4 nm. The thickness may be determined by methods known in the art, for example measured from transmission electron microscopy (TEM) images.

The magnetic nanoparticle may be ferromagnetic material. The term “ferromagnetic” will be understood to refer to a permanent magnetic material (a material which can be magnetised by an external magnetic field and remain magnetised after that magnetic field is removed) in which all the magnetic moments (dipoles) are aligned in the same direction. Typically, when a magnetic material is decreased to low nanoscale size (eg below about 50 nm size), these materials can lose their ability to exhibit ferromagnetic behaviour at room temperature and instead exhibit superparamagnetic behaviour. Advantageously, as shown in the Examples, the present inventors have surprisingly found that the magnetic nanoparticles described herein are capable of exhibiting ferromagnetic behaviour at room temperature without application of an external magnetic field. Accordingly, in some embodiments, the magnetic nanoparticle is ferromagnetic. In some embodiments, the magnetic nanoparticle is ferromagnetic at room temperature (eg a temperature between about 20° C. to about 27° C.) in the absence of an external magnetic field.

The magnetic nanoparticle described herein may be characterised by one or more properties, including magnetic and theranostic properties. Each magnetic material of the magnetic nanoparticle and/or the core, shell and nanoparticle dimensions may be suitably selected to tune one or more desired properties of the magnetic nanoparticle.

Magnetic properties may include coercivity (H_{C}) and magnetisation saturation (M_{S}), which may be determined by methods known in the art, for example by measuring magnetisation-field (M-H) loops (also called hysteresis curves).

Magnetic properties may also include blocking temperature (T_{B}), which may be determined by methods known in the art, for example by measuring zero field cooling (ZFC) and field cooling (FC) curves. In some embodiments, the magnetic nanoparticle has a blocking temperature of at least about 270 K, at least about 280 K, at least about 290 K, or at least about 300 K.

Theranostic properties may include specific absorption rate (SAR), which may be determined by methods known in the art, for example from thermal activation plots. In some embodiments, the magnetic nanoparticle has a specific absorption rate of at least about 1.5 W/g, at least about 2.0 W/g, at least about 2.5 W/g, at least about 3.0 W/g, at least about 3.5 W/g, at least about 4.0 W/g, at least about 4.5 W/g, at least about 5.0 W/g, at least about 6.0 W/g, at least about 7.0 W/g, at least about 8.0 W/g, at least about 9.0, or at least about 10.0 W/g, measured at a field strength of 5 V at a field frequency of 176 kHz. In some embodiments, the magnetic nanoparticle has a specific absorption rate of at least about 1 W/g, at least about 2 W/g, at least about 3 W/g, at least about 4 W/g, at least about 5 W/g, at least about 6 W/g, at least about 7 W/g, at least about 8 W/g, at least about 9 W/g, at least about 10 W/g, at least about 11 W/g, at least about 12 W/g, at least about 13 W/g, at least about 14 W/g, at least about 15 W/g, at least about 16 W/g, at least about 18 W/g, at least about 20 W/g, at least about 22 W/g, at least about 24 W/g, at least about 26 W/g, at least about 28 W/g, or at least about 30 W/g, measured at a field strength of 15 V at a field frequency of 176 kHz. In some embodiments, the magnetic nanoparticle has a specific absorption rate of at least about 5 W/g, at least about 6 W/g, at least about 7 W/g, at least about 8 W/g, at least about 9 W/g, at least about 10 W/g, at least about 12 W/g, at least about 14 W/g, at least about 15 W/g, at least about 16 W/g, at least about 18 W/g, at least about W/g, at least about 20 W/g, at least about 25 W/g, at least about 30 W/g, at least about 40 W/g, at least about 50 W/g, at least about 60 W/g, or at least about 70 W/g, measured at a field strength of 15 V at a field frequency of 176 KHz.

### Preparation of Magnetic Nanoparticles

The present disclosure provides a method of preparing a magnetic nanoparticle having a core-multishell structure. The method comprises the steps of:
providing a nanoparticle core comprising a first magnetic material;
heating a first mixture comprising the nanoparticle core, one or more second magnetic material precursors and one or more surfactants in a dispersing medium to a temperature not exceeding about 290° C. such that a first shell comprising a second magnetic material is deposited on the nanoparticle core to provide a core-shell nanoparticle comprising a single shell; and
heating a second mixture comprising the core-shell nanoparticle, one or more third magnetic material precursors and one or more surfactants in a dispersing medium to a temperature not exceeding about 290° C. such that a second shell comprising a third magnetic material is deposited on the first shell to provide a core-multishell nanoparticle comprising two shells;
   wherein the second magnetic material has a different coercivity (H_{C}) to the first magnetic material, and the third magnetic material has a different H_{C} to the second magnetic material.

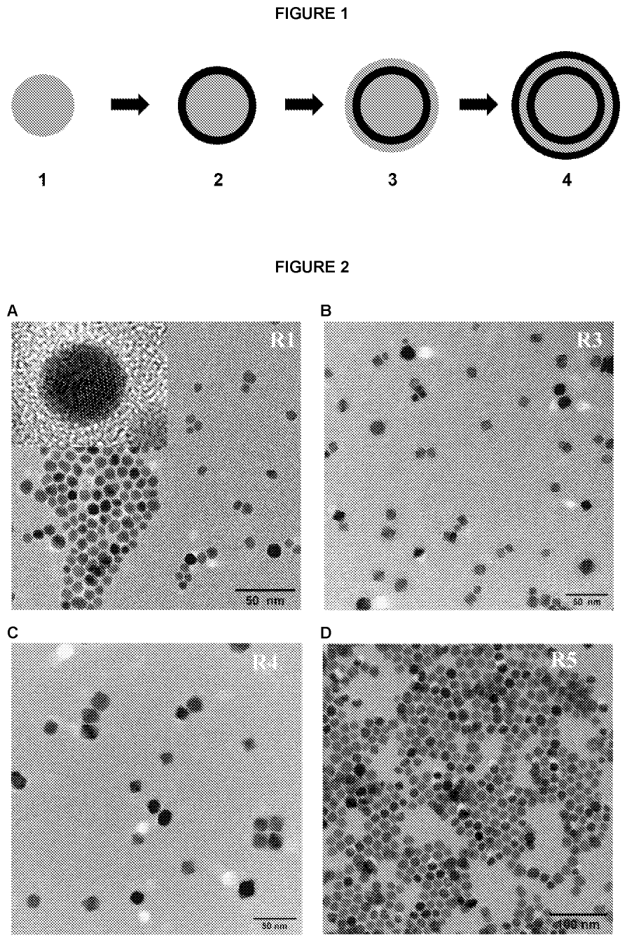

FIG. 1 is a schematic representation depicting the formation of an illustrative magnetic nanoparticle having a core-multishell structure comprising three shells. The magnetic nanoparticle is prepared from a core (1), onto which are subsequently deposited a first shell, a second shell and a third shell to provide a core-shell structure comprising a single shell (2), a core-multishell structure comprising two shells (3) and finally a core-multishell structure comprising three shells (4).

Advantageously, the method disclosed herein is capable of providing magnetic nanoparticles having a core-multishell structure which comprises two or more shells. Further, the method may advantageously prevent inter diffusion of atomic species between each shell and the core, thereby avoiding phase impurities in the shell and core magnetic materials. The inventors have surprisingly found that heating to a temperature not exceeding 290° C. may reduce the decomposition of the one or more magnetic materials to be deposited in the heating step, and may also avoid the separate nucleation of nanoparticles from these magnetic materials (ie the formation of separate nanoparticles instead of depositing to form a shell).

The magnetic nanoparticle provided by the method may comprise a third shell comprising a fourth magnetic material, wherein the fourth magnetic material has a different H_{C} to the third magnetic material.

Accordingly, in some embodiments, the method further comprises the step of:
heating a third mixture the core-multishell nanoparticle comprising two shells, one or more fourth magnetic material precursors and one or more surfactants in a dispersing medium to a temperature not exceeding about 290° C. such that a third shell comprising a fourth magnetic material is deposited on the second shell to provide a core-multi-shell nanoparticle comprising three shells;
   wherein the third magnetic material has a different H_{C} to the second magnetic material.

The magnetic nanoparticle provided by the method may optionally comprise one or more further shells (in addition to the second shell or the third shell, if present), said one or more further shells surrounding an immediately preceding shell, said one or more further shells each independently comprising a magnetic material having a different H_{C} to magnetic material in an immediately preceding shell.

Accordingly, in some embodiments, the method further comprises the steps of:
(i) heating a further mixture comprising the core-multishell nanoparticle, one or more further magnetic material precursors and one or more surfactants in a dispersing medium to a temperature not exceeding about 290° C. such that a further shell comprising a further magnetic material is deposited on the outermost shell of the core-multishell nanoparticle; and
(ii) optionally repeating step (i) one or more times;
   to provide one or optionally more further shells each independently comprising a further magnetic material on the core-multishell nanoparticle;
   wherein the one or optionally more additional shells each have a different H_{C} to the preceding shell.

In these embodiments, the step (ii) may be repeated as many times so as to provide the desired number of shells in the provided magnetic nanoparticle.

In some embodiments, the first mixture is prepared by the following steps:
combining the nanoparticle core and the one or more surfactants in the dispersing medium to provide a first dispersion; and
combining the first dispersion with the one or more second magnetic material precursors to provide the first mixture.

In some embodiments, the second mixture is prepared by the following steps:
combining the core-shell nanoparticle comprising a single shell and the one or more surfactants in the dispersing medium to provide a second dispersion; and
combining the second dispersion with the one or more third magnetic material precursors to provide the second mixture.

In embodiments where the magnetic nanoparticle comprises a third shell, the third mixture may prepared by the following steps:
combining the core-multishell nanoparticle comprising two shells and the one or more surfactants in the dispersing medium to provide a third dispersion; and
combining the third dispersion with the one or more fourth magnetic material precursors to provide the third mixture.

In embodiments where the magnetic nanoparticle comprises one or more further shells, each further mixture may be independently prepared by the following steps:
combining the core-multishell nanoparticle and the one or more surfactants in the dispersing medium to provide a further dispersion; and
combining the further dispersion with the one or more further magnetic material precursors to provide the further mixture.

In these embodiments, each combining step may independently comprise mixing the respective components, for example by stirring and/or by sonicating. Advantageously, mixing may allow for improved dispersion of the respective components in the respective dispersing medium and may avoid aggregation of these components.

The temperature in each of the heating steps does not exceed about 290° C. It will be understood that the temperature of each of the heating steps are independent of each other, that is, the temperature of each heating step may be the same or may be different. Each heating step may independently comprise heating from room temperature (eg a temperature between about 20° C. to about 27° C.). In some embodiments, each heating step independently comprises heating to a temperature not exceeding about 290° C., about 285° C., about 280° C., about 275° C., about 270° C., about 260° C., about 250° C., about 240° C., about 230° C., about 210° C., about 200° C., or about 190° C. In some embodiments, each heating step independently comprises heating to a temperature of about 180° C., about 190° C., about 200° C., about 210° C., about 220° C., about 230° C., about 240° C., about 250° C., about 260° C., about 270° C., about 280° C., or about 290° C. Any two values may be combined to form a temperature range provided the range is within 180° C. to about 290° C., for example a temperature ranging from about 190° C. to about 290° C. or from about 180° C. to about 250° C.

Each heating step of the method described herein may independently comprise heating to a temperature not exceeding the boiling point of the respective dispersing medium (ie the dispersing medium used in that heating step), provided that the temperature does not exceed 290° C. By way of example, in embodiments where the dispersing medium of a heating step comprises phenyl ether (which has a boiling point of about 258° C.), that heating step may comprise heating to a temperature not exceeding the boiling point of phenyl ether (eg a temperature not exceeding about 258° C.). In embodiments where the dispersing medium comprises more than one organic liquid, the heating step may comprise heating to a temperature not exceeding the boiling point of the organic liquid in the dispersing medium with the lowest boiling point. It will be understood that in embodiments where the dispersing medium of a heating step has a boiling point above 290° C. (eg benzyl ether, which has a boiling point of about 298° C.), the temperature of the heating step does not exceed 290° C. The inventors have surprisingly found that heating to a temperature not exceeding the boiling point of the respective dispersing medium (while also not exceeding 290° C.) may reduce the decomposition of the one or more magnetic materials to be deposited in the heating step, and may also avoid the separate nucleation of nanoparticles from these magnetic materials (ie the formation of separate nanoparticles instead of depositing to form a shell).

Each heating step may independently comprise mixing the respective mixture (ie the mixture heated in that heating step) by methods known in the art, for example by stirring.

The rate of heating in each heating step may be controlled. In some embodiments, each heating step independently comprises heating at a rate of from about 3° C./min to about 10° C./min, preferably at a rate of about 5° C./min. In some embodiments, each heating step independently comprises heating a rate of from about 3° C./min, about 4° C./min, about 5° C./min, about 6° C./min, about 7° C./min, about 8° C./min, about 9° C./min, or about 10° C./min. Any two values can be combined to form a range provided the heating rate is within 3° C./min to 10° C./min, for example a heating rate of from about 4° C./min to about 7° C./min. The present inventors have surprisingly found that gradually (rather than rapidly) increasing the temperature to the target temperature may avoid the separate nucleation of nanoparticles from the one or more magnetic materials to be deposited magnetic materials (ie the formation of separate nanoparticles instead of depositing to form a shell).

Once the desired temperature of a heating step is reached, that temperature may be maintained for a duration sufficient to deposit the respective shell (ie the shell deposited in that heating step). In some embodiments, each heating step independently comprises heating at the temperature for a duration of from about 20 minutes to about 45 minutes, preferably for a duration of about 30 minutes. In some embodiments, each heating step independently comprises heating at the temperature for about 20 minutes, about 25 minutes, about 30 minutes, about 35 minutes, about 40 minutes, or about 45 minutes. Any two values can be combined to form a range provided the duration is within 20 minutes to 45 minutes, for example a duration of from about 25 minutes to about 35 minutes.

The method described herein may further comprise, after any heating step, a step of cooling the respective mixture (ie the mixture heated in that heating step). In some embodiments, the mixture is cooled to room temperature (a temperature between about 20° C. to about 27° C.).

The method may further comprise, after any heating step, a step of washing the respective mixture (ie the mixture heated in that heating step) with a desolvating agent. The washing step may precipitate the respective nanoparticle (ie the core-shell nanoparticle or the core-multishell nanoparticle provided in that heating step) from the mixture. In some embodiments, the desolvating agent comprises one or more polar liquids, for example one or more of acetone, isopropanol, methanol and ethanol. In some embodiments, the desolvating agent comprises or consists of ethanol. In some embodiments, the desolvating agent further comprises one or more non-polar liquids, for example one or more of hexane, heptane, cyclohexane and toluene, in addition to the one or more polar liquids. In some embodiments, the desolvating agent comprises acetone and one or both of hexane and toluene. In embodiments where the desolvating agent comprises one or more polar liquids and one or more non-polar liquids, the desolvating agent may comprise the one or more polar liquids and the one or more non-polar liquids in a volume ratio of from about 1:1 to about 3:1, preferably from about 1:1 to about 2:1.

The method may further comprise, after any heating step, a step of separating the respective nanoparticle (ie the core-shell nanoparticle or the core-multishell nanoparticle provided in that heating step) from the respective mixture (ie the mixture heated in that heating step). The respective nanoparticles may be separated by methods known in the art, for example by using a magnet and by centrifugation.

The method may further comprise, after any heating step, a step of drying the respective nanoparticle (ie the core-shell nanoparticle or the core-multishell nanoparticle provided in that heating step). The respective nanoparticles may be dried by methods known in the art, for example by air-drying.

The method may further comprise a step of functionalising the outermost shell (ie the surface) of the core-multishell nanoparticle with one or more functional moieties to provide a functionalised magnetic nanoparticle. This step is typically conducted after all the desired shells have been deposited onto the core-multishell nanoparticle. This step may alternatively be described as a step of attaching one or more functional moieties to the outermost shell of the core-multishell nanoparticle to provide a functionalised magnetic nanoparticle. Also described herein is a method of preparing a functionalised nanoparticle, the method comprising providing the magnetic nanoparticle described herein, and functionalising the outermost shell (ie the surface) of the magnetic nanoparticle with one or more functional moieties to provide the functionalised nanoparticles. Any suitable functional moiety known in the art can be used, including those described herein.

The functionalising step may be carried out by methods known in the art for providing the desired functional moiety. Typically, the functionalising step comprises physical functionalisation and/or covalent functionalisation. Physical functionalisation (or physical attachment) involves attaching one or more functional moieties to the core-multishell nanoparticle by electrostatic interaction, for example by adsorption of negatively charged molecules to the positively charged core-multishell nanoparticle surface and vice versa. Covalent functionalisation (or covalent attachment) involves attaching one or more functional moieties to the core-multishell nanoparticle by a covalent bond, for example by an amide or carbonyl bond or any other suitable covalent bond for attaching the functional moiety to the surface of the core-multishell nanoparticle. Accordingly, in some embodiments, the functionalising (or attaching) step comprises attaching at least one of the one or more functional moieties to the outermost shell of the core-multishell nanoparticle by a covalent bond. Additionally, or alternatively, in some embodiments, the functionalising (or attaching) step comprises attaching at least one of the one or more functional moieties to the outermost shell of the core-multishell nanoparticle by electrostatic interaction. The type of attachment may be suitably selected depending on the functional moiety and its intended function. For example, covalent functionalisation may be used to attach polymers to the core-shell nanoparticles, which may allow for long term stability of the nanoparticles in aqueous solution without aggregation.

In some embodiments, the step of providing the nanoparticle core comprises:
heating an initial mixture comprising one or more first magnetic material precursors and one or more surfactants in a dispersing medium up to a refluxing temperature (ie a temperature exceeding the boiling point of the dispersing medium) to provide the core nanoparticles.

In these embodiments, one or more of the following may apply:
the heating step comprises mixing the initial mixture by methods known in the art, for example by stirring;
the heating step comprises heating at a rate from about 3° C./min to about 10° C./min, preferably at a rate of about 5° C./min;
the heating step comprises heating for a duration of from about 1 hour to about 5 hours, preferably from about 2 hours to about 3 hours;

The step of providing the nanoparticle core may further comprise one or more of the following steps:
cooling the initial mixture, preferably to room temperature (a temperature between about 20° C. to about 27° C.);
washing the initial mixture with a desolvating agent, for example as described herein;
separating the core nanoparticle from the initial mixture, for example as described herein;
drying the core nanoparticle, for example as described herein.

The first magnetic material of the nanoparticle core may comprise one or more of cobalt ferrite, barium ferrite, strontium ferrite, manganese ferrite, nickel ferrite, manganese oxide and iron oxide. In preferred embodiments, the first magnetic material comprises one or more hard phase magnetic materials, for example one or more of cobalt ferrite, barium ferrite and strontium ferrite.

For each heating step, each of the respective one or more magnetic material precursors (ie the one or more magnetic material precursors used in that heating step) may be any suitable precursor for providing the desired one or more magnetic materials for each respective shell or the core as described herein. In some embodiments, each of the one or more respective magnetic material precursors independently comprise an iron precursor and one or more of a cobalt precursor, a barium precursor, a strontium precursor, a manganese precursor and a nickel precursor. Suitable magnetic material precursors include metal complexes (eg metal complexes comprising an acetylacetonate anion, acac, ligand), where the metal is a metal present in the magnetic material. In preferred embodiments, the one or more magnetic material precursors comprise one or more metal acetylacetonate complexes. Examples of suitable iron precursors include iron (III) acetylacetonate (Fe(acac)₃). Examples of suitable cobalt precursors include cobalt acetylacetonate (Co(acac)₂). Examples of suitable barium precursors include barium acetylacetonate (Ba(acac)₂). Examples of suitable strontium precursors include strontium acetylacetonate (Sr(acac)₂). Examples of suitable manganese precursors include manganese acetylacetonate (Mn(acac)₂). Examples of suitable nickel precursors include nickel acetylacetonate (Ni(acac)₂).

The respective one or more magnetic material precursors may each be independently be provided in amounts suitable to provide the desired one or more magnetic materials for each respective shell or the core as described herein. In embodiments where each of the one or more respective magnetic material precursors independently comprise an iron precursor and one or more of a cobalt precursor, a barium precursor, a strontium precursor, a manganese precursor and a nickel precursor, the molar ratio of the iron precursor to the one or more of a cobalt precursor, a barium precursor, a strontium precursor, a manganese precursor and a nickel precursor may be about 1:1 to about 3:1, preferably about 2:1.

The respective one or more magnetic material precursors may be independently provided in amounts suitable to provide a shell comprising the desired magnetic material that surrounds the immediately preceding shell (or in the case of the first shell, the core nanoparticle). In some embodiments, the molar ratio of nanoparticle core to the one or more second magnetic material precursors is about 1:3 to about 1:1, preferably about 1:2. In some embodiments, the molar ratio of nanoparticle core to the one or more second magnetic material precursors is about 1:3 to about 1:1, preferably about 1:2. In embodiments where the provided core-multishell nanoparticle comprises three shells, the molar ratio of the core-multishell nanoparticle comprising two shells to the one or more third magnetic material precursors may be about 1:3 to about 1:1, preferably about 1:2. In embodiments where the provided core-multishell nanoparticle comprises one or more further shells (in addition to the second shell or the third shell, if present), for each further shell, the molar ratio of the core-multishell nanoparticle to the one or more further magnetic material precursors may be about 1:3 to about 1:1, preferably about 1:2. The present inventors have surprisingly found that these ratios may advantageously allow for control of shell thickness.

For each heating step, the respective dispersing mediums (ie the dispersing medium of that heating step) may comprise any suitable organic liquid capable of dispersing the components therein, including those known in the art. Examples of suitable dispersing mediums include one or more of ethers, hydrocarbons including saturated and unsaturated hydrocarbons (preferably unsaturated hydrocarbons such as alkanes), aromatics, and optionally other substituted organic liquids.

In some embodiments, each of the dispersing mediums independently comprise one or more non-polar organic liquids, for example one or more non-polar ethers, hydrocarbons including saturated and unsaturated hydrocarbons (preferably unsaturated hydrocarbons such as alkanes), aromatics, and optionally other substituted organic liquids. In some embodiments, each of the dispersing mediums independently comprise one or more of benzyl ether, phenyl ether, hexadecane and octadecene. In preferred embodiments, each of the dispersing mediums independently comprise benzyl ether. In some embodiments, each of the dispersing mediums may independently consist of a single organic liquid, preferably a single non-polar organic liquid more preferably selected from one of benzyl ether, phenyl ether, hexadecane and octadecene, preferably benzyl ether. In this context, “consist(s) of” in respect of a dispersing medium will be understood to mean that dispersing medium does not include any further organic liquids, that is, the dispersing medium includes only the single organic liquid (eg benzyl ether) as the dispersing medium. The present inventors have surprisingly found that the heating steps may be performed using a single organic liquid (rather than a mixture of organic liquids) as the dispersing medium.

Additionally, or alternatively, in some embodiments, each of the dispersing mediums may independently have a minimum boiling point of greater than about 180° C., for example greater than about 190° C., greater than about 200° C., greater than about 210° C., greater than about 220° C., greater than about 230° C., greater than about 240° C., greater than about 250° C., greater than about 260° C., greater than about 270° C., or greater than about 280° C. Accordingly, by way of example, in some embodiments, each of the dispersing mediums independently comprise one or more non-polar organic liquids having a minimum boiling point of greater than about 180° C.

For each heating step, the respective one or more surfactants (ie the one or more surfactants of that heating step) may comprise any suitable surfactant, including those known in the art. Examples of suitable surfactants include cationic surfactants (eg amines), anionic surfactants (eg carboxylic acids) and non-ionic surfactants (eg alcohols) which comprise at least one saturated or unsaturated hydrocarbon chain greater than 8 carbon atoms in the chain, including greater than 10 carbon atoms, greater than 12 carbon atoms, and greater than 14 carbon atoms. In some embodiments, each of the one or more surfactants may comprise one or more of oleic acid, oleylamine, 1,2-hexadecanediol and hexadecylamine, preferably one or more of oleic acid, oleylamine and 1,2-hexadecanediol. In embodiments where the heating step results in deposition of a shell, each of the one or more surfactants may independently consist of a single surfactant, preferably a single surfactant selected from one of oleic acid, oleylamine, 1,2-hexadecanediol and hexadecylamine, preferably oleic acid. In this context, “consist(s) of” will be understood to mean that the one or more surfactants include only a single surfactant (eg oleic acid). The present inventors have surprisingly found that these heating steps may be performed using a single surfactant (rather than multiple surfactants).

The magnetic nanoparticle provided by the method described herein may have one or more of the following properties:
the nanoparticle core has an average diameter of from about 5 nm to about 15 nm, preferably about 6 to about 10 nm;
each shell independently has an average thickness of from about 1 nm to about 5 nm, preferably about 2 nm to about 4 nm;
the magnetic nanoparticle has an average diameter of less than about 40 nm, preferably less than about 30 nm, more preferably less than about 25 nm, even more preferably less than about 20 nm.

The present disclosure also provides the magnetic nanoparticle prepared by the method disclosed herein. The magnetic nanoparticle prepared by the method may be the magnetic nanoparticle as defined in any one or more of the herein disclosed embodiments.

### Applications

The magnetic nanoparticles described herein and produced by the methods described herein may be used in a variety of applications. Illustrative examples include as permanent magnets (particularly rare-earth free permanent magnets), in information storage, in biomedical applications (eg magnetic hyperthermia, non-invasive diagnostic imaging), in energy applications (eg energy conversion and storage, including in motors, generators and batteries), in separation technologies (eg treatment of water such as sea water or waste water to remove organic contaminants, oil and/or heavy metals), and in environmental applications. Advantageously, the properties of the magnetic nanoparticle may be tuned (eg based on the overall size, shell thickness and number of shells in the core-multishell structure) to be suitable for use in certain applications. By way of example, magnetic nanoparticles having moderate coercivity and moderate magnetic moment may be suitable for use in magnetic hyperthermia; magnetic nanoparticles having high magnetic moment and low coercivity may be suitable for use as imaging contrasting agents in magnetic resonance imaging; magnetic nanoparticles having high coercivity and high magnetic moment may be suitable for use as high energy permanent magnets; and magnetic nanoparticles that are ferromagnetic at room temperature may be suitable for use in data storage.

Accordingly, the present disclosure provides the use of the magnetic nanoparticles disclosed herein or prepared by the method disclosed herein for any one or more of the herein disclosed applications. The present disclosure also provides the magnetic nanoparticle disclosed herein or prepared by the method disclosed for use in any one or more of the herein disclosed applications. The present disclosure further provides a method of using the magnetic nanoparticles disclosed herein or prepared by the method disclosed herein for any one or more of the herein disclosed applications.

As disclosed herein and shown in the Examples, the magnetic nanoparticles disclosed herein may be useful in magnetic hyperthermia. In this context, the term “magnetic hyperthermia” will be understood to refer to a medical treatment, typically for treating cancer, which involves the generation of heat from the magnetic nanoparticles upon application of an external alternating magnetic field for targeted therapeutic heating of body tissue, including tumours.

Accordingly, the present disclosure provides a method of magnetic hyperthermia, the method comprising:
administering the magnetic nanoparticle disclosed herein or prepared by the method disclosed herein to a patient, and
exposing the magnetic nanoparticle to an alternating magnetic field.

The present disclosure also provides use of the magnetic nanoparticle disclosed herein or prepared by the method disclosed herein for magnetic hyperthermia. The present disclosure further provides the magnetic nanoparticle disclosed herein or prepared by the method disclosed herein for use in magnetic hyperthermia.

The present disclosure additionally provides a permanent magnet comprising the magnetic nanoparticle disclosed herein or prepared by the method disclosed herein. The permanent magnet may be suitable for use in energy-generating devices, for example generators and motors, for storing and converting energy. Accordingly, the present disclosure also provides the use of the permanent magnet for storing and/or converting energy. The present disclosure further provides the permanent magnet for use in storing and/or converting energy.

The magnetic nanoparticle may be provided in any form suitable for an intended application. By way of example, for biomedical applications, the magnetic nanoparticle may be provided in the form of a pharmaceutical composition comprising the magnetic nanoparticle and a pharmaceutically acceptable carrier, which may be for delivery to a patient or a body tissue of a patient.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

### EXAMPLES

The present disclosure will be further described by way of non-limiting examples. It will be understood to persons skilled in the art of the present disclosure that many modifications may be made without departing from the spirit and scope of the present disclosure.

### Example 1. Preparation of Magnetic Nanoparticles Having Core-Multishell Structure

The synthesis of MnFe₂O₄@CoFe₂O₄@MnFe₂O₄@CoFe₂O₄ (core@shell@shell@shell) nanoparticles is provided below. A similar procedure was also used to prepare CoFe₂O₄@MnFe₂O₄@CoFe₂O₄, CoFe₂O₄@BaFe₁₂O₁₉@CoFe₂O₄, BaFe₁₂O₁₉@CoFe₂O₄@BaFe₁₂O₁₉, CoFe₂O₄@SrFe₁₂O₁₉@CoFe₂O₄, SrFe₁₂O₁₉@CoFe₂O₄@SrFe₁₂O₁₉ and CoFe₂O₄@BaFe₁₂O₁₉@CoFe₂O₄@BaFe₁₂O₁₉ nanoparticles, using the appropriate precursors to provide the respective magnetic materials present in each core and shell layer of the respective magnetic nanoparticles.

Step 1: Synthesis of CoFe₂O₄ Core Nanoparticles

Cobalt ferrite (CoFe₂O₄) nanoparticle seeds (core nanoparticles) were prepared by mixing 2 mmol of iron (III) acetylacetonate (Fe(acac)₃), 1 mmol of cobalt (II) acetylacetonate (Co(acac)₂), 10 mmol of 1,2-hexadecanediol, 6 mmol of oleic acid, 6 mmol of oleylamine and 20 mL of benzyl ether in a 100 mL round bottom flask using a magnetic stirrer at approximately 1000 rpm on a Heidoph Hei-Tec Magnetic Stirrer. A Eurotherm Heat Controller/Indicator was programmed to heat the mixture to 200° C. at a ramp rate of 5° C./min, hold at 200° C. for 120 minutes, heat to 300° C., hold for 60 minutes and then cool to approximately room temperature (˜25° C.) to end the program. The obtained mixture resembled a black-brown dispersion. Following the reflux reaction, the solution was then pipetted into a 200 ml conical flask. 40 mL toluene and 80 mL acetone (1:2 toluene to acetone) was added as a desolvating agent. The solution was placed on a solid magnet to facilitate the separation of nanoparticles from the solution. Nanoparticles were collected and dried with air. The final nanoparticles were in a black solid form.

Step 2: Synthesis of First Shell (CoFe₂O₄@MnFe₂O₄)

0.5 mmol of the obtained CoFe₂O₄ nanoparticle seeds from Step 1 were dispersed in 5 mL benzyl ether and 600 μL of oleic acid. The mixture was subsequently sonicated for 5 minutes. An additional 15 mL benzyl ether was added to disperse and transfer the CoFe₂O₄ seeds into a round bottom flask with a magnetic stirrer. 0.3 mmol of manganese (II) acetylacetonate (Mn(acac)₂) was combined with 0.67 mmol of Fe(acac)₃ and added to the CoFe₂O₄ mixture. The consequent mixture was magnetically stirred at approximately 1000 rpm. The heat controller was programmed to heat the mixture to 290° C. at a ramp rate of 5° C./min, hold for 30 minutes and then cool to approximately room temperature (˜25° C.). After the reaction was completed, the solution was then pipetted into a 200 ml conical flask with 20 mL hexane and 40 mL acetone (about 1:2 hexane to acetone). The solution was placed on a solid magnet to facilitate the separation of nanoparticles from the solution. The supernatant was discarded, and the CoFe₂O₄@MnFe₂O₄ nanoparticles were collected and air-dried.

Step 3: Synthesis of Second Shell (CoFe₂O₄@MnFe₂O₄@CoFe₂O₄)

A similar procedure to Step 2 was used to provide a second shell comprising CoFe₂O₄ on the first shell of the CoFe₂O₄@MnFe₂O₄ nanoparticles obtained in Step 2. 0.5 mmol of CoFe₂O₄@MnFe₂O₄ core-shell nanoparticles was dispersed in 5 mL benzyl ether and 600 μL oleic acid. The mixture was sonicated for 5 minutes. 15 mL of benzyl ether was added to disperse and transfer the CoFe₂O₄@MnFe₂O₄ nanoparticles into a round bottom flask with a magnetic stirrer. 0.3 mmol of Co(acac)₂ and 0.67 mmol Fe(acac)₃ was added to the CoFe₂O₄@MnFe₂O₄ mixture. The mixture was magnetically stirred and heated with the same parameters described in Step 2. The obtained nanoparticles were then pipetted into a 20 0 ml conical flask with 20 mL hexane and 40 mL acetone (about 1:2 hexane to acetone) and placed on a solid magnet to facilitate the separation of the nanoparticles. The supernatant was discarded and the CoFe₂O₄@MnFe₂O₄@CoFe₂O₄ nanoparticles were collected and air-dried.

Step 4: Synthesis of Third Shell (CoFe₂O₄@MnFe₂O₄@CoFe₂O₄@MnFe₂O₄)

A similar procedure to Step 2 was used to provide a third shell comprising MnFe₂O₄, where 0.5 mmol of the CoFe₂O₄@MnFe₂O₄@CoFe₂O₄ nanoparticles obtained in Step 3 was used as the precursor to provide CoFe₂O₄@MnFe₂O₄@CoFe₂O₄@MnFe₂O₄ nanoparticles.

### Synthesis of Functionalised Nanoparticles

CoFe₂O₄@MnFe₂O₄ and CoFe₂O₄@MnFe₂O₄@CoFe₂O₄@MnFe₂O₄ nanoparticles were functionalised by using previously described methods (Pham et al, International Journal of Molecular Science, 2018, Volume 19, Issue 1, 205-228; Pham et al, International Journal of Nanomedicine, 2017, Volume 12, 899-909). In brief, single-shell CoFe2O4@MnFe2O4 nanoparticles obtained in step 2 and triple shell CoFe2O4@MnFe2O4@CoFe2O4@MnFe2O4 nanoparticles obtained in step 3 were functionalised with methoxy-PEG (MPEG) via reversible addition fragmentation transfer (RAFT) using block copolymers comprising a RAFT block, a phosphate block and an MPEG block. The phosphate block allow for the polymer to anchor to the iron oxide on the surface of the nanoparticles. The MPEG block can allow for stabilisation of the nanoparticles. The RAFT-MPEG block-copolymer functionalisation can attach onto and stabilise the nanoparticles, even after cellular uptake.

### Example 2. Morphological and Structural Characterisation of Nanoparticles

Transmission electron microscopy (TEM) was used to determine the morphology, size, and chemical composition of the CoFe₂O₄ seed nanoparticles and CoFe₂O₄@MnFe₂O₄, CoFe₂O₄@MnFe₂O₄@CoFe₂O₄ and CoFe₂O₄@MnFe₂O₄@CoFe₂O₄@MnFe₂O₄ nanoparticles (NPs) obtained in Example 1. TEM images were obtained using a JEOL JEM-2100 electron microscope operating at 200 kV (performed at the Australian Centre for Microscopy and Microanalysis, University of Sydney, Sydney). For TEM imaging, 10 microliter of nanoparticles was deposited on TEM copper grid (300-mesh) containing a thin carbon coated farmover transparent film and left to dry until complete evaporation of the solvent (about 30 min). The nanoparticles were dispersed in toluene.

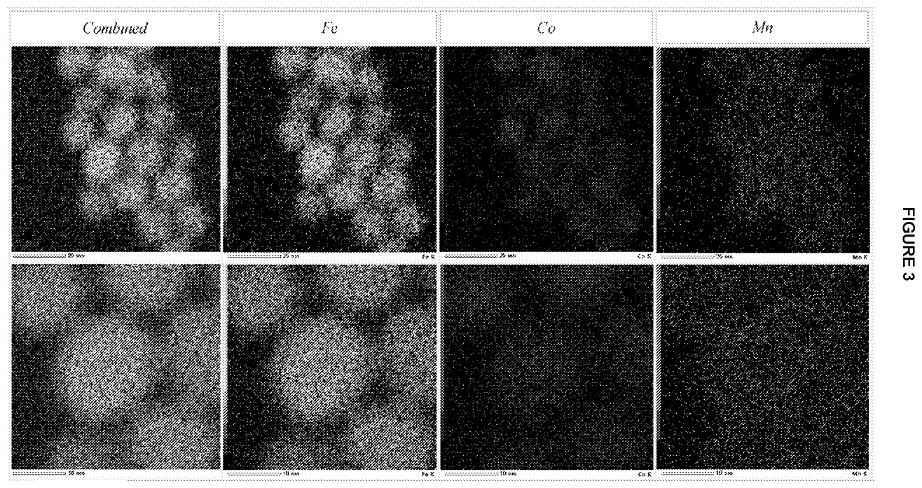

Table 1 summarises the nanoparticle sizes as determined from representative TEM images via Image J software analysis. FIG. 2 shows TEM images of CoFe₂O₄ seed nanoparticles (FIG. 2A), CoFe₂O₄@MnFe₂O₄ nanoparticles (FIG. 2B), CoFe₂O₄@MnFe₂O₄@CoFe₂O₄ nanoparticles (FIG. 2C) and CoFe₂O₄@MnFe₂O₄@CoFe₂O₄@MnFe₂O₄ nanoparticles (FIG. 2D). The inset of FIG. 2A is a high resolution TEM image of the CoFe₂O₄ seed sample.

TABLE 1
Size of nanoparticles

| | Mean | Median | Mode | Minimum | Maximum | Standard |
|---|---|---|---|---|---|---|
| | diameter | diameter | diameter | diameter | diameter | deviation |
| Sample | (nm) | (nm) | (nm) | value (nm) | value (nm) | (σ) |
| CoFe₂O₄ seed | 8.96 | 8.79 | 10.57 | 5.60 | 14.87 | 1.59 |
| CoFe₂O₄ seed | 8.90 | 8.67 | 9.24 | 5.05 | 14.85 | 1.85 |
| CoFe₂O₄@ | 12.70 | 12.31 | 12.03 | 6.54 | 22.58 | 3.09 |
| MnFe₂O₄ NP | | | | | | |
| CoFe₂O₄@ | 16.87 | 17.17 | 18.61 | 9.24 | 23.49 | 2.51 |
| MnFe₂O₄@ | | | | | | |
| CoFe₂O₄ NP | | | | | | |
| CoFe₂O₄@ | 19.03 | 19.18 | 20.24 | 11.07 | 31.08 | 2.46 |
| MnFe₂O₄@ | | | | | | |
| CoFe₂O₄@ | | | | | | |
| MnFe₂O₄ NP | | | | | | |

Two CoFe₂O₄ seed nanoparticle samples were prepared and evaluated. The two seed samples showed similar size distributions and had a mean diameter of 8.96 nm and of 8.90 nm, respectively. Based on TEM images, the two seed samples exhibited relatively spherical morphologies and appeared relatively homogeneous and monodispersed (see FIG. 2A). The high resolution TEM image shown in the inset of FIG. 2A indicates the formation of single crystalline nanoparticles.

The two CoFe₂O₄ seed nanoparticle samples were used for the synthesis of the first-, second- and third-shell structures. The size of the nanoparticles increased as the number of the shells around the core increased. The mean particle diameters of the first-shell CoFe₂O₄@MnFe₂O₄ nanoparticles and second shell CoFe₂O₄@MnFe₂O₄@CoFe₂O₄ nanoparticles were 12.70 nm and 16.87 nm, respectively, and the size distributions of were broader than the two core samples. The third-shell CoFe₂O₄@MnFe₂O₄@CoFe₂O₄@MnFe₂O₄ nanoparticles had a mean diameter of 19.03 nm and had higher monodispersity and homogeneity in the size distribution.

Energy dispersive spectrometer (EDS) mapping was performed for the final CoFe2O4@MnFe2O4@CoFe2O4@MnFe2O4 nanoparticles to determine the composition and location of its elemental constituents. EDS mapping was performed using the JEOL JEM2100 electron microscope equipped with EDS detector for the analysis. Prior to the analysis, the elements were selected in the software based on the elements present in the nanoparticle sample (eg Fe, Co, Mn) and signals from these elements were collected using the EDS detector after the electron beam hit the sample.

FIG. 3 shows the elemental mapping of iron, cobalt and manganese across the nanoparticle, appearing white in the labelled panel. All elements were present across the entire nanoparticle. The composite image shows a homogenous distribution of the different elements. EDS mapping indicated a lower signal from manganese component than to iron and cobalt constituents. This is due to the presence of substantially less manganese component compared to iron and cobalt constituents in the initial growth solution as well as thin shell after the synthesis of the nanoparticles.

X-ray photoelectron microscopy (XPS) was performed to determine the composition of the CoFe₂O₄ seed nanoparticles, CoFe₂O₄@MnFe₂O₄, CoFe₂O₄@MnFe₂O₄@CoFe₂O₄ and CoFe₂O₄@MnFe₂O₄@CoFe₂O₄@MnFe₂O₄ nanoparticles, and MPEG-functionalised CoFe₂O₄@MnFe₂O₄ and CoFe₂O₄@MnFe₂O₄@CoFe₂O₄@MnFe₂O₄ nanoparticles. Table 2 summarises the atomic amounts as determined by XPS. The XPS results further confirm the formation of core-multishell magnetic nanoparticles.

TABLE 2
Atomic amounts of elements in nanoparticles

| Sample | 0 1 s | C 1 s | Fe 2 p | Fe 2 p_{1/2} | Co 2 p | Co 2 p_{1/2} | Mn 2 p | Mn 2 p_{1/2} |
|---|---|---|---|---|---|---|---|---|
| CoFe₂O₄ seed | 57.47% | 16.80% | 12.07% | 7.16% | 5.01% | — | — | — |
| CoFe₂O₄@ | 51.37% | 23.60% | 9.61% | 8.10% | 2.70% | 0.46% | 1.15% | 1.02% |
| MnFe₂O₄ NP | | | | | | | | |
| CoFe₂O₄@ | 36.27% | 33.07% | 17.04% | 7.47% | 5.19% | 1.73% | 2.88% | 0.78% |
| MnFe₂O₄@ | | | | | | | | |
| CoFe₂O₄ NP | | | | | | | | |
| CoFe₂O₄@ | 47.65% | 27.11% | 10.89% | 8.48% | 3.45% | 0.47% | 0.91% | 1.04% |
| MnFe₂O₄@ | | | | | | | | |
| CoFe₂O₄@ | | | | | | | | |
| MnFe₂O₄ NP | | | | | | | | |
| Fn/CoFe₂O₄@ | 52.58% | 27.25% | 7.95% | 4.04% | 5.29% | 1.77% | 0.67% | 0.49% |
| MnFe₂O₄ NP | | | | | | | | |
| Fn/CoFe₂O₄@ | 40.25% | 36.69% | 7.21% | 6.02% | 3.37% | 0.37% | 0.71% | 0.81% |
| MnFe₂O₄@ | | | | | | | | |
| CoFe₂O₄@ | | | | | | | | |
| MnFe₂O₄ NP | | | | | | | | |

Comparative X-Ray Diffraction (XRD) was performed to compare the structures of the CoFe₂O₄ seed nanoparticles and the CoFe₂O₄@MnFe₂O₄, CoFe₂O₄@MnFe₂O₄@CoFe₂O₄ and CoFe₂O₄@MnFe₂O₄@CoFe₂O₄@MnFe₂O₄ nanoparticles. XRD analysis was performed using a STOE STANDI P (STOE, Darmstadt, Germany) with a Molybdenum source (λ=0.7107 Å) available. The samples were first powered and loaded onto 0.5 mm capillaries and inserted onto the sample holder. The sample was then calibrated to be centered and parallel to the beam and was subsequently analyzed for 30 minutes at an angle range of 5-55°. For data analysis, the samples were processed using the PDF-4+ program and peak matched to an existing database of XRD patterns.

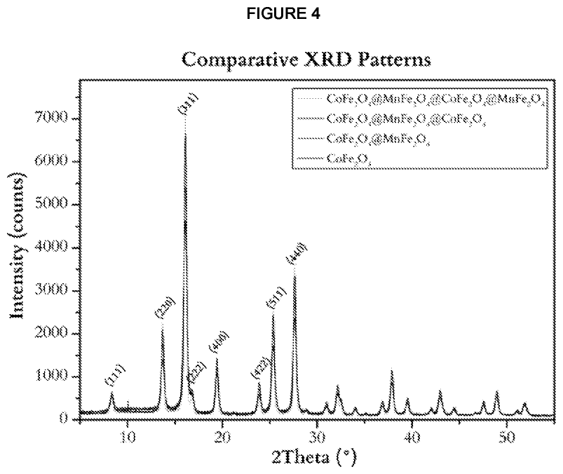

FIG. 4 shows the comparative XRD patterns for the four samples. As indicated by the comparative XRD pattern, all the samples have a similar cubic structure, characterised predominantly by the CoFe₂O₄ constituent component. The diffraction patterns all map well onto the JCPDS CoFe₂O₄ card 00-022-1086 (International Centre for Diffraction Data database), indicating that all the samples exhibit a major spinel structure phase and a Fd-3m (227) space group symmetry. This indicates that the nanoparticles may have an inherently face-centred cubic structure.

### Example 3. Magnetic Characterisation Nanoparticles

### Magnetic Hysteresis

The magnetic properties including coercivity and magnetic saturation of the CoFe₂O₄ seed nanoparticles and the CoFe₂O₄@MnFe₂O₄, CoFe₂O₄@MnFe₂O₄@CoFe₂O₄ and CoFe₂O₄@MnFe₂O₄@CoFe₂O₄@MnFe₂O₄ nanoparticles were determined by measuring hysteresis curve (Magnetization, M vs applied field, H) at room temperature (300 K) using vibrating sample magnetometry.

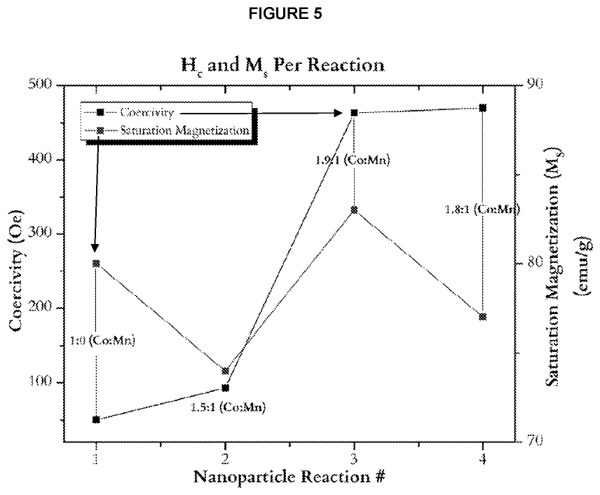

The coercivity (H_{C}) and saturation magnetisation (M_{S}) results are shown in FIG. 5. Generally, coercivity increased with the addition of further shells onto the initial CoFe₂O₄ seed. The CoFe₂O₄ seed nanoparticles exhibited a coercivity (H_{C}) of 50 Oe. The intermediate single-shell CoFe₂O₄@MnFe₂O₄ and double-shell CoFe₂O₄@MnFe₂O₄@CoFe₂O₄ nanoparticles exhibited increasing coercivity of 93 Oe and 463 Oe respectively. Finally, the triple-shell CoFe₂O₄@MnFe₂O₄@CoFe₂O₄@MnFe₂O₄ nanoparticles had a coercivity of 470 Oe.

The results indicate a global increase in coercivity, which may be predominantly due to the addition of the highly coercive CoFe₂O₄ phase. This, however, may be moderated by the low coercivity (and high saturation magnetisation) of the MnFe₂O₄ phase, enabling structures with coercive properties which are intermediate of the hard/soft phases. This may indicate why the CoFe₂O₄ seed and single-shell CoFe₂O₄@MnFe₂O₄ nanoparticle have similar coercivity values given that that both these samples have approximately the same % Co, and similarly why double-shell CoFe₂O₄@MnFe₂O₄@CoFe₂O₄ and triple shell CoFe₂O₄@MnFe₂O₄@CoFe₂O₄@MnFe₂O₄ nanoparticles have similar coercivity values. given that that both these samples have approximately the same % Co. On the other hand, when there is an increase in the % Co with the addition of a CoFe₂O₄ phase, (eg from CoFe₂O₄@MnFe₂O₄ to CoFe₂O₄@MnFe₂O₄@CoFe₂O₄), there is a substantial increase in the coercivity, contributed from the hard CoFe₂O₄ component. The increased in the magnetic exchange coupling between hard/soft/hard/soft phases may also contribute to the increased coercivity in CoFe₂O₄@MnFe₂O₄@CoFe₂O₄@MnFe₂O₄ nanoparticles.

The results also indicate a general decrease in the saturation magnetisation due to the addition of the hard CoFe₂O₄ phase. The XPS, TEM and EDS analyses indicate that the CoFe₂O₄ phase predominates, which may indicate that there is a lesser contribution from the high M₅ (and consequently lower H_{C}) of the soft MnFe₂O₄ phase. Therefore, the energy product of the final CoFe₂O₄@MnFe₂O₄@CoFe₂O₄@MnFe₂O₄ nanostructures would have a higher H_{C} and lower M₅, due to the greater contribution of the CoFe₂O₄ phase.

An overall trend of the coercivity and saturation magnetisation may be gleaned from FIG. 5. The coercivity increases with the deposition of additional phases. Further, the saturation magnetisation is dependent on the ratio of MnFe₂O₄:CoFe₂O₄. Each nanoparticle will exhibit properties which are intermediate of the constituent hard and soft phases. Given the greater CoFe₂O₄ ratio across the four nanoparticle samples, coercivity values remain high. The magnetic properties appear to be predominantly contributed by the hard cobalt ferrite phase. Further, the saturation magnetisation of the sample fluctuates with the presence of the softer manganese ferrite phase.

### Zero Field Cooling/Field Cooling Curves

Zero field cooling (ZFC) and field cooling (FC) can be used to determine the dependence of the magnetic moment of a sample on the temperature. From the ZFC/FC curves, one is able to derive the blocking temperature (T_{B}) at the point at which the ZFC and FC curves diverge or the point at which the derivative of the difference between the ZFC and FC (d(FC-ZFC)/dT) curve is maximum. The blocking temperature provides indication of the ferroparamagnetic (or superparamagnetic) behaviour at room temperature (300K). The blocking temperature represents the point where thermal fluctuations within the nanoparticles overcomes the energy barrier to moment reversal, thereby causing the spontaneous, rapid flipping of the moment vector. Here, when the system energy (from thermal fluctuations, defined by k_{B}T) is equal to or greater than the energy barrier (Eₐ=KᵤV), then there is net zero magnetism: the nanoparticles are superparamagnetic. Therefore, if T_{B}<300 K, the material would exhibit superparamagnetic behaviours at room temperature. Further, if T_{B}>300 K, the material would retain their ferromagnetic alignment even at room temperature. The blocking temperature is defined by the following equation:

$${T}_{B} = {K}_{u} V / {k}_{B} ln ( {t}_{m} / {t}_{0} )$$

where Kᵤ is the uniaxial anisotropy constant, V is the nanoparticle volume, k_{B} is the Boltzmann's constant, τₘ is the measurement time, and τ₀ is a constant related to gyromagnetic precision and may be considered to be of the order of 10⁻⁹-10⁻¹⁰ s.

The blocking temperature thus represents the ratio of the anisotropic energy (KᵤV) to the system energy (k_{B}T). The blocking temperature is therefore directly related to the size of the nanoparticles. As the nanoparticles increase in size from the deposition of additional layers, there should be an expected size contribution to the blocking temperature. Coercivity also has the following relationship with the blocking temperature:

$${H}_{C} = {{H}_{0} ( 1 - T / {T}_{B} )}^{1 / 2}$$

where H₀ is the coercivity at (T=0K), T_{B} is the superparamagnetic blocking temperature, and T is the system temperature.

The blocking temperature is therefore related to the coercivity in such a way that an increase in the blocking temperature can imply an increase in the coercivity. Given that the blocking temperature represents the point at which the system energy (from k_{B}T) supersedes the anisotropic energy barrier (KᵤV), a lower anisotropic barrier (or higher system energy) can imply greater spin agitation and therefore lower coercivity. This threshold point is determined by the blocking temperature. Thus, a higher blocking temperature may generally imply a higher coercivity.

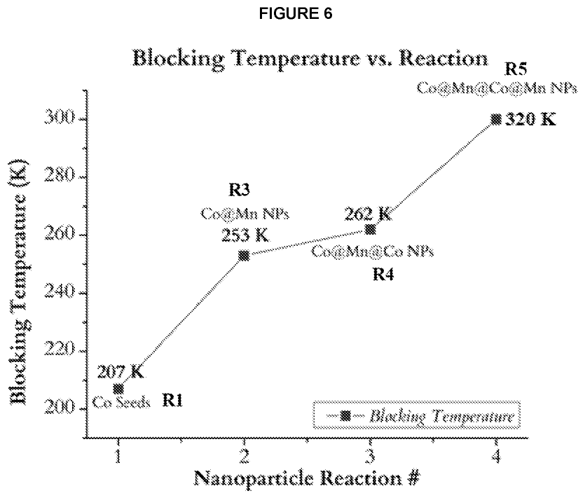

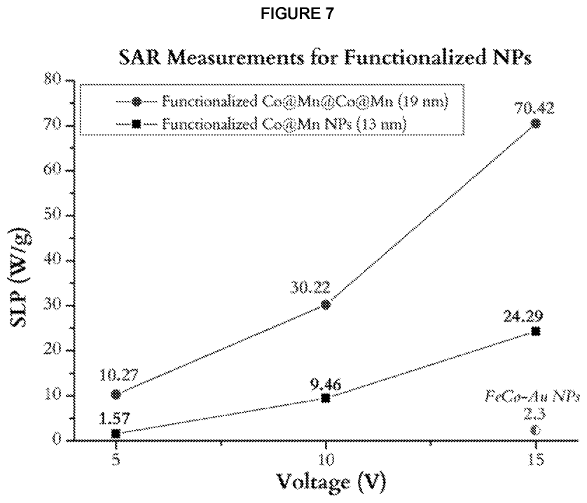

FIG. 6 illustrates the blocking temperature T_{B} for the CoFe₂O₄ seed nanoparticles and the CoFe₂O₄@MnFe₂O₄, CoFe₂O₄@MnFe₂O₄@CoFe₂O₄ and CoFe₂O₄@MnFe₂O₄@CoFe₂O₄@MnFe₂O₄ nanoparticles. For the CoFe₂O₄ seed nanoparticles, the blocking temperature was determined to be 207 Oe. Single-shell CoFe2O4@MnFe2O4 nanoparticles showed increase in the blocking temperature to 253 K, which is expected due to the additional layer of soft magnetic phase MnFe₂O₄. The added soft MnFe₂O₄ phase was expected to increase the anisotropy of the system from the interfacial exchange interactions between the two phases. Further, it would increase the total volume of the nanoparticle, thereby increasing the T_{B}. For the double-shell CoFe₂O₄@MnFe₂O₄@CoFe₂O₄ nanoparticles, the blocking temperature was determined to be 262 K, which is a slight increase from the single-shell structure. For the triple-shell CoFe₂O₄@MnFe₂O₄@CoFe₂O₄@MnFe₂O₄ nanoparticles, the blocking temperature was determined to be 300 K. Therefore, the triple-shell nanoparticles can be said to be ferromagnetic at room temperature. The CoFe₂O₄@MnFe₂O₄@CoFe₂O₄@MnFe₂O₄ nanoparticles, due to the coupling of the phases, may exhibit high exchange interactions (at the coupled interfaces) and therefore higher coercivities from the exchange ‘locked’ spins. This in turn may result in a higher blocking temperature, as a greater system energy would be required to destabilise the exchange-coupled spins (from the hard/soft coupled phases). Further, there is an additional size contribution from the exterior shell. Thus, the blocking temperature appears to correlate well with the associated characteristics of the CoFe₂O₄@MnFe₂O₄@CoFe₂O₄@MnFe₂O₄ nanoparticles.

### Example 4. Heating Efficiency of Magnetic Nanoparticles Having Core-Multishell Structure for Magnetic Hyperthermia

Measurement of heat generation was performed under a field frequency of 176 kHz, which is relatively close to the clinically tolerable threshold. The clinical application of the nanoparticles limited the field strength (H) and frequency (f) of the magnetic field that can be applied. Across the scientific literature to date, the H·F limitations vary between 4.5×10⁸ (A/m·s) (or 5.625×10⁶ Oe/s) and 8.5×10⁸ (A/m·s) (or 10.625×10⁶ Oe/s).

The specific absorption rate (SAR) was measured using the following formula:

$$SAR = ( {C}_{fluid} \cdot {V}_{s} \cdot dT / dt ) / m$$

where C_{fluid} is the concentration of the nanoparticles in the sample, Vₛ is the volume of the sample (500 μL), m is the mass of the nanoparticles in the sample, and dT/dt is the change in temperature over time. The SAR was determined at 5, 10 and 15V at a fixed field frequency of 176 kHz, corresponding to a field of 7, 15 and 21 mT respectively. The study was performed for the two functionalised samples, namely MPEG-functionalised CoFe₂O₄@MnFe₂O₄ and CoFe₂O₄@MnFe₂O₄@CoFe₂O₄@MnFe₂O₄ nanoparticles.

The SAR results are shown in FIG. 6. At the field parameters, the SAR of the functionalised single core-shell nanoparticles were determined to be 1.57 W/g, 9.46 W/g and 24.29 W/g at 5, 10 and 15 V respectively. There was a substantial increase in the SAR values of the functionalised triple-shell nanoparticles, which exhibited values of 10.27 W/g, 30.22 W/g and 70.42 W/g at 5, 10 and 15 V respectively. The deposition of the two additional phases resulted in an approximate threefold increase in the SAR values. The increased SAR for the functionalised triple-shell CoFe₂O₄@MnFe₂O₄@CoFe₂O₄@MnFe₂O₄ nanoparticles is indicative of the greater energy product, (BH)_{Max}, from the additional CoFe₂O₄ and MnFe₂O₄ phases. This may be in part due to the increased coercivity from the magnetically hard cobalt phase. However, the addition of the soft MnFe₂O₄ phase would contribute to a higher saturation magnetization, which may also increase the energy product.

The SAR results were compared to those obtained for Fe(Co)—Au core-shell nanoparticles described in Kline et al. (Journal of Magnetism and Magnetic Materials, Volume 321, Issue 10, May 2009, Pages 1525-1528). Kline et al demonstrated that Fe(Co)—Au core-shell nanoparticles (12 nm) produced SAR values of 2.3 W/g at frequencies of 191 kHz. Therefore, the obtained SAR values of the functionalised CoFe₂O₄@MnFe₂O₄ and functionalised CoFe₂O₄@MnFe₂O₄@CoFe₂O₄@MnFe₂O₄ nanoparticles demonstrate a ten to thirty-fold increase in the heat output from the iron-based nanoparticles of Kline et al. It is noted that are reports in the literature showing higher value of SAR than to the functionalised CoFe₂O₄@MnFe₂O₄ and functionalised CoFe₂O₄@MnFe₂O₄@CoFe₂O₄@MnFe₂O₄ nanoparticles described herein, however, these measurements were performed using non-clinical parameters (high frequency and field amplitude).

1. A magnetic nanoparticle having a core-multishell structure, said core-multishell structure comprising:
a core, said core comprising a first magnetic material;
a first shell, said first shell surrounding the core, said first shell comprising a second magnetic material;
a second shell, said second shell surrounding the first shell, said second shell comprising a third magnetic material; and
optionally one or more further shells, said one or more further shells surrounding an immediately preceding shell, said one or more further shells each independently comprising a magnetic material;
wherein the second magnetic material has a different coercivity (H_{C}) to the first magnetic material, the third magnetic material has a different H_{C} to the second magnetic material, and the optional one or more further shells each comprise magnetic material having a different H_{C} to magnetic material in an immediately preceding shell.

2. The magnetic nanoparticle according to claim 1, wherein the first shell, the second shell and each optional further shell alternate between comprising magnetic material having a lower or higher H_{C} than an immediately preceding shell or, in the case of the first shell, the core.

3. The magnetic nanoparticle according to claim 1, wherein the first magnetic material and the third magnetic material are the same.

4. The magnetic nanoparticle according to claim 1, wherein the magnetic nanoparticle further comprises a third shell comprising a fourth magnetic material, wherein the fourth magnetic material has a different H_{C} to the third magnetic material.

5. The magnetic nanoparticle according to claim 1, wherein the first magnetic material and the second magnetic material independently comprise one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide.

6. (canceled)

7. The magnetic nanoparticle according to claim 1 to 6, having a core-multishell structure selected from:
CoFe₂O₄@MnFe₂O₄@CoFe₂O₄ (core@shell@shell)
CoFe₂O₄@BaFe₁₂O₁₉@CoFe₂O₄ (core@shell@shell)
BaFe₁₂O₁₉@CoFe₂O₄@BaFe₁₂O₁₉ (core@shell@shell)
CoFe₂O₄@SrFe₁₂O₁₉@CoFe₂O₄ (core@shell@shell)
SrFe₁₂O₁₉@CoFe₂O₄@SrFe₁₂O₁₉ (core@shell@shell)
CoFe₂O₄@MnFe₂O₄@CoFe₂O₄@MnFe₂O₄ (core@shell@shell@shell)
CoFe₂O₄@BaFe₁₂O₁₉@CoFe₂O₄@BaFe₁₂O₁₉ (core@shell@shell@shell)

8. The magnetic nanoparticle according to claim 1, wherein one or more of the following apply:
the core has an average diameter of from about 5 nm to about 15 nm, preferably about 6 to about 10 nm;
each shell independently has an average thickness of from about 1 nm to about 5 nm, preferably about 2 nm to about 4 nm;
the magnetic nanoparticle has an average diameter of less than about 40 nm, preferably less than about 30 nm, more preferably less than about 25 nm, even more preferably less than about 20 nm.

9. The magnetic nanoparticle according to claim 1, wherein the magnetic nanoparticle further comprises one or more functional moieties attached to the outermost shell of the core-multishell structure.

10. (canceled)

11. The magnetic nanoparticle according to claim 1, wherein the magnetic nanoparticle exhibits ferromagnetism at room temperature without application of an external magnetic field.

12. The magnetic nanoparticle according to claim 1, wherein the magnetic nanoparticle has a specific absorption rate (SAR) of at least about 20 W/g, measured at a field strength of 15 V at a field frequency of 176 kHz.

13. A method of preparing a magnetic nanoparticle having a core-multishell structure, the method comprising:
providing a nanoparticle core comprising a first magnetic material;
heating a first mixture comprising the nanoparticle core, one or more second magnetic material precursors and one or more surfactants in a dispersing medium to a temperature not exceeding about 290° C. such that a first shell comprising a second magnetic material is deposited on the nanoparticle core to provide a core-shell nanoparticle comprising a single shell; and
heating a second mixture comprising the core-shell nanoparticle, one or more third magnetic material precursors and one or more surfactants in a dispersing medium to a temperature not exceeding about 290° C. such that a second shell comprising a third magnetic material is deposited on the first shell to provide a core-multishell nanoparticle comprising two shells;
wherein the second magnetic material has a different coercivity (H_{C}) to the first magnetic material, and the third magnetic material has a different H_{C} to the second magnetic material.

14. The method according to claim 13, wherein the method further comprises:
heating a third mixture the core-multishell nanoparticle comprising two shells, one or more fourth magnetic material precursors and one or more surfactants in a dispersing medium to a temperature not exceeding about 290° C. such that a third shell comprising a fourth magnetic material is deposited on the second shell to provide a core-multi-shell nanoparticle comprising three shells;
wherein the third magnetic material has a different H_{C} to the second magnetic material.

15. The method according to claim 13, wherein the method further comprises:
(i) heating a further mixture comprising the core-multishell nanoparticle, one or more further magnetic material precursors and one or more surfactants in a dispersing medium to a temperature not exceeding about 290° C. such that a further shell comprising a further magnetic material is deposited on the outermost shell of the core-multishell nanoparticle; and
(ii) optionally repeating step (i) one or more times;
to provide one or optionally more further shells each independently comprising a further magnetic material on the core-multishell nanoparticle;
wherein the one or optionally more additional shells each have a different H_{C} to the preceding shell.

16. (canceled)

17. (canceled)

18. The method according to claim 13, wherein the first magnetic material comprises one or more of cobalt ferrite, barium ferrite, strontium ferrite, manganese ferrite, nickel ferrite, manganese-zinc ferrite, zinc ferrite, zinc-iron ferrite, zinc-nickel-ferrite, manganese oxide and iron oxide.

19. (canceled)

20. The method according to claim 13, wherein each of the one or more respective magnetic material precursors independently comprise an iron precursor and one or more of a cobalt precursor, a barium precursor, a strontium precursor, a manganese precursor and a nickel precursor.

21. The method according to claim 20, wherein the molar ratio of the iron precursor to the one or more of a cobalt precursor, a barium precursor, a strontium precursor, a manganese precursor and a nickel precursor is about 1:1 to about 3:1.

22. The method according to claim 13, wherein one or more of the following apply:
the molar ratio of nanoparticle core to the one or more second magnetic material precursors is about 1:3 to about 1:1;
the molar ratio of nanoparticle core to the one or more second magnetic material precursors is about 1:3 to about 1:1;
the molar ratio of the core-multishell nanoparticle comprising two shells to the one or more third magnetic material precursors is about 1:3 to about 1:1;
the molar ratio of the core-multishell nanoparticle to the one or more further magnetic material precursors is about 1:3 to about 1:1.

23. The method according to claim 13, wherein each of the dispersing mediums independently comprise one or more non-polar organic liquids.

24. (canceled)

25. (canceled)

26. The method according to claim 13, wherein each of the one or more surfactants independently comprise one or more cationic surfactants, anionic surfactants and non-ionic surfactants comprising at least one saturated or unsaturated hydrocarbon chain greater than 8 carbon atoms.

27. (canceled)

28. (canceled)

29. The method according to claim 13, wherein the method further comprises functionalising the outermost shell of the outermost shell of the core-multishell structure with one or more functional moieties.

30. A method of magnetic hyperthermia comprising:
administering the magnetic nanoparticle according to claim 1 or prepared by the method of claim 13 to a patient, and
exposing the magnetic nanoparticle to an alternating magnetic field.
